(19) United States
(12) Patent Application Publication
(10) Pub. No.: US 2025/0388843 A1
(43) Pub. Date: Dec. 25, 2025

(54) MEDIUM EXCHANGE SYSTEM FOR CELL CULTURE BOTTLE CAPABLE OF PREVENTING MUTUAL CONTAMINATION

(71) Applicant: CellFabs Inc., Tainan City (TW)

(72) Inventors: Yun-Shu CHIOU, Tainan City (TW); Tzu-Yang HUANG, Tainan City (TW); Shang-Chih LIN, Tainan City (TW)

(73) Assignee: CellFabs Inc., Tainan City (TW)

(21) Appl. No.: 18/749,300

(22) Filed: Jun. 20, 2024

(51) Int. Cl. C12M 1/00; C12M 1/24; C12M 3/00; C12M 3/06

(52) U.S. Cl. CPC C12M 29/00; C12M 23/08; C12M 23/46; C12M 27/16

(57) ABSTRACT

A medium exchange system for cell culture bottles that prevents mutual contamination includes a medium exchange device, which has a stepper motor connected to a linear slide above. The first linear slide is driven to rotate by the stepper motor and has a first slider and a second slider, each having a lower clamping arm and an upper clamping arm. A medium exchange container includes a medium exchange cylinder attached to an extension tube on an end thereof, while another end thereof is inserted by a push rod. The lower clamping arm is adapted to grip the medium exchange cylinder, and the upper clamping arm is adapted to grip the push rod. Both the clamping arms move towards a cell culture bottle, allowing the extension tube to penetrate into the cell culture bottle for medium exchange. The used medium exchange container can be replaced by using the medium exchange device.

### BACKGROUND OF THE DISCLOSURE

### 1. Field of the Disclosure

The present invention relates generally to a medium exchange system for cell culture bottles, and more particularly to a medium exchange system capable of exchanging medium automatically and preventing mutual contamination.

### 2. Description of the Prior Art

Conventional cell culture methods involve placing obtained cells in cell culture bottles and soaking them in culture medium for preservation in an environment conducive to cell growth. During cell cultivation in the incubator, it is necessary to periodically check the cell status and quantity in the bottles. Prior to reaching the harvest criteria, it is necessary to exchange the medium in the cell culture bottles to provide fresh or adjusted culture medium to continue supplying the nutrients required by the cells. However, upon closer examination of the aforementioned conventional structure, it is evident that there are some shortcomings. The main reason for these shortcomings is as follows: conventional medium exchange devices typically consist of multiple tubes connected to different culture medium reservoirs and waste reservoirs. When removing the old culture medium from the cell culture bottle, the tube connected to the waste reservoir is inserted into the bottle for suction. When adding new culture medium, the tube connected to the culture medium reservoir is inserted into the bottle for filling. Since these tubes are reused, they are likely to accumulate culture medium from multiple cell culture bottles on their surfaces. This not only leads to cross-contamination between multiple tubes but also results in cross-contamination between different cell culture bottles. Therefore, the conventional medium exchange method may significantly reduce the success rate of cell cultivation. This is the technical problem that the present invention aims to address.

In view of this, the inventor, with many years of experience in the manufacturing, development, and design of related products, meticulously designed and carefully evaluated solutions to the above-mentioned objectives. As a result, the present invention with practical utility has been achieved.

### SUMMARY OF THE DISCLOSURE

The technical problem that the present invention aims to solve is to address the aforementioned shortcomings of existing technology by providing a medium exchange system for cell culture bottle capable of preventing mutual contamination.

A medium exchange device, which has a stepper motor fixed to a base thereof, wherein the stepper motor is connected to a first linear slide located above, and is adapted to drive the first linear slide to rotate; the medium exchange device is provided with a first slider and a second slider in a slidable manner at the first linear slide; the first slider is provided with a lower clamping arm, and the second slider is provided with a upper clamping arm. A medium exchange container, which includes a medium exchange cylinder, a push rod, and an extension tube, wherein the medium exchange cylinder has a communicating tube formed at an end thereof; the extension tube fits around the communicating tube; a wide opening is formed at another end of the medium exchange cylinder, whereby the push rod enters the medium exchange cylinder through the wide opening; the lower clamping arm is adapted to clamp the medium exchange cylinder, and the upper clamping arm is adapted to clamp the push rod; the lower clamping arm and the upper clamping arm are both adapted to move toward a cell culture bottle, so that the extension tube is adapted to enter the cell culture bottle for medium exchange, and the medium exchange device is adapted to replace the used medium exchange container.

In an embodiment, the medium exchange system further includes a medium providing bottle, wherein the medium providing bottle has an opening provided thereon, and the medium exchange container is adapted to be placed into the medium providing bottle by passing the extension tube through the opening; when only the upper clamping arm moves upward to move the push rod and the medium exchange cylinder to extract new culture medium in the medium providing bottle, the new culture medium is filled into the medium exchange cylinder.

In an embodiment, the medium exchange system further includes a recycling bottle, wherein the recycling bottle has a bottle opening provided thereon, and the medium exchange container is adapted to be placed into the recycling bottle by passing the extension tube into the bottle opening; when only the upper clamping arm moves downward to move the push rod and the medium exchange cylinder, the old culture medium is injected into the recycling bottle.

In an embodiment, the medium exchange system further includes a gripping mechanism, wherein the gripping mechanism includes a swing-functioning motor, a swing base, a extend-functioning motor, and a gripping arm, wherein the swing-functioning motor is fixed to the base of the medium exchange device, and the swing-functioning motor is linked to the swing base to drive the swing base; the extend-functioning motor is provided at the swing base, and the extend-functioning motor is linked to the gripping arm to drive the gripping arm, so that the gripping arm is adapted to grip the cell culture bottle in this manner, and the swing-functioning motor is adapted to shake the cell culture bottle for mixing culture medium.

In an embodiment, the medium exchange system further includes a transporting mechanism, wherein the transporting mechanism is provided with a second linear slide; a third linear slide is provided between the swing base and the extend-functioning motor; the second linear slide and the third linear slide are perpendicular to each other; the third linear slide drives the gripping arm to move to the second linear slide for gripping and placing the cell culture bottle.

In an embodiment, a transporting platform is provided at the second linear slide in a slidable manner; the transporting platform is provided with a miniature motor, and the miniature motor is linked to a flipping board to drive the flipping board; the cell culture bottle is adapted to lie on the flipping board for transportation; when the cell culture bottle is transported to the gripping arm, the miniature motor drives the flipping board to erect the cell culture bottle.

In an embodiment, a fourth linear slide is provided between the transporting platform and the miniature motor, and the fourth linear slide and the second linear slide are perpendicular to each other, so that the flipping board and the cell culture bottle are both movable toward the gripping arm.

In an embodiment, the medium exchange system further includes a container exchange mechanism, wherein the container exchange mechanism is provided on a fifth linear slide in a slidable manner, and the container exchange mechanism is provided with a plurality of clamps, wherein the clamps are adapted to clamp the medium exchange cylinder of the medium exchange container, and provide the medium exchange container to the lower clamping arm and the upper clamping arm of the medium exchange device.

In an embodiment, the clamps are provided at a center post in a radial pattern, so that all of the medium exchange containers surround the center post; a driving motor is provided at the center post, wherein the driving motor is adapted to rotate the center post, whereby the clamps and the lower clamping arm are adapted to deliver the medium exchange container to each other.

In an embodiment, the container exchange mechanism has a drip receiving disk provided by the fifth linear slide, and at least an UV lamp is provided both at the drip receiving disk and the base of the medium exchange device.

The main purpose of the present invention is to utilize the rotation of the stepper motor to drive the first linear slide in circular motion, enabling the lower clamping arm and the upper clamping arm to rotate to specified positions for gripping the new and discarded medium exchange containers. Additionally, they rotate to predetermined positions for extracting old culture medium for recycling and extracting new culture medium for filling. The lower clamping arm holds the medium exchange cylinder of the medium exchange container, while the upper clamping arm holds the push rod. When both the lower clamping arm and the upper clamping arm move up and down simultaneously, they synchronize the vertical movement of the medium exchange container. Furthermore, when only the upper clamping arm moves, the push rod is actuated to extract or discharge the culture medium. This achieves the purpose of replacing the culture medium. The used medium exchange container can be directly discarded and replaced with a new one, further preventing mutual contamination between the medium providing bottle, the recycling bottle, and the cell culture bottle, thus combining automatic medium exchange with the practical effect of eliminating contamination risks.

Other objectives, advantages, and novel features of the present invention will become more apparent from the following detailed description and accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of the medium exchange device of the present invention;

FIG. 2 is a schematic view showing the operation of the medium exchange device of the present invention;

FIG. 3 is a schematic view showing the process of extracting culture medium from the cell culture bottle;

FIG. 4 is a schematic view showing the process of disposing of old culture medium into the recycling bottle;

FIG. 5 is a schematic view showing the process of exchanging the medium exchange container;

FIG. 6 is a schematic view showing the process of extracting new culture medium from the medium providing bottle;

FIG. 7 is a schematic view showing the process of injecting the culture medium into the cell culture bottle;

FIG. 8 is a perspective view of the gripping mechanism and the transporting mechanism of the present invention;

FIG. 9 is a schematic view showing the process of clamping the cell culture bottle;

FIG. 10 is a schematic view showing the process of shaking the cell culture bottle;

FIG. 11 is a perspective view of the medium exchange device and the container exchange mechanism of the present invention; and

FIG. 12 is a top view of the medium exchange device and the container exchange mechanism of the present invention.

### DETAILED DESCRIPTION

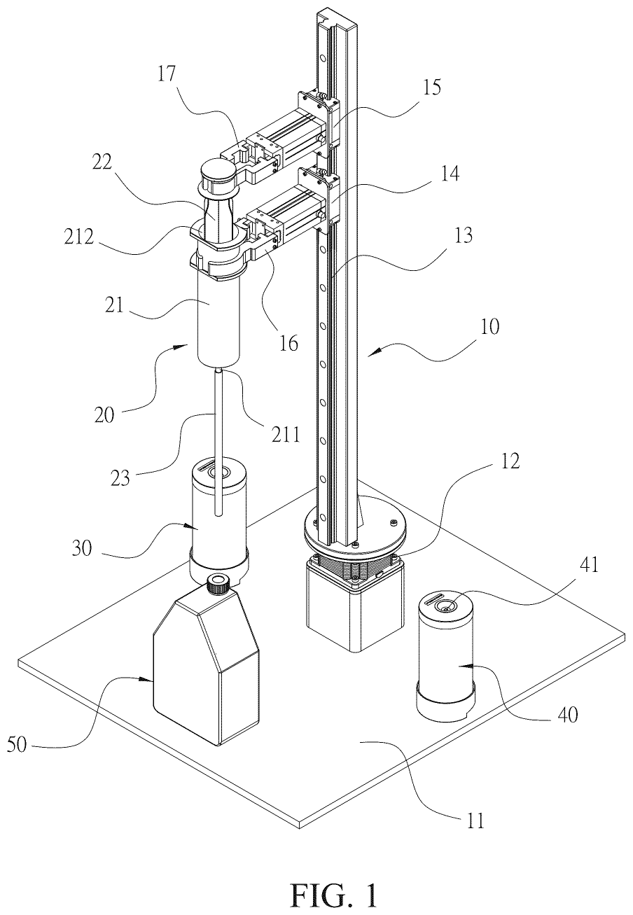

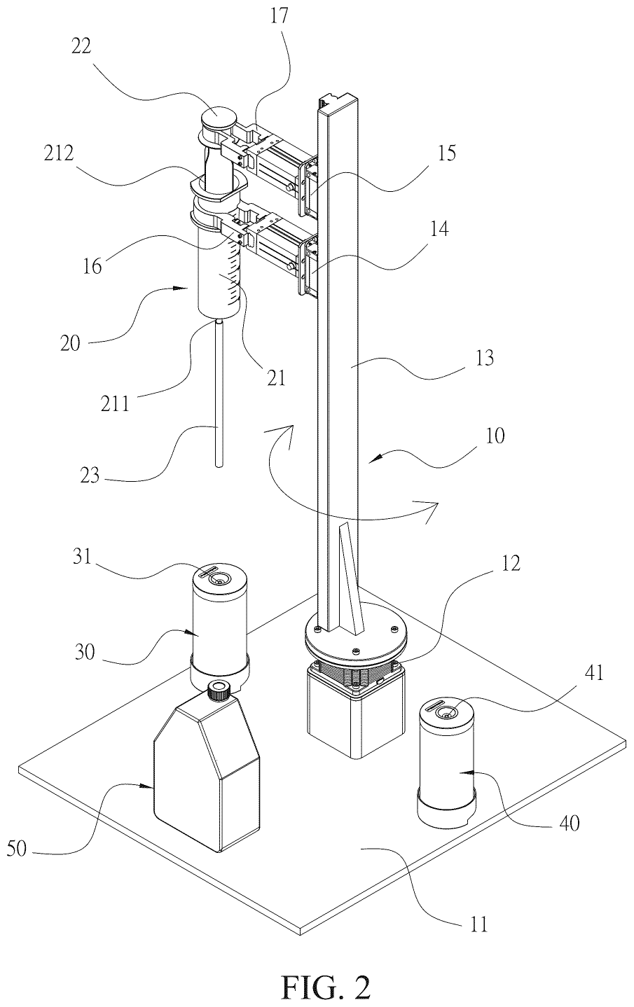

FIG. 1 and FIG. 2 illustrate a medium exchange system for cell culture bottles, which is capable of preventing mutual contamination. The medium exchange system includes a medium exchange device 10, a medium exchange container 20, a medium providing bottle 30, and a recycling bottle 40. The medium exchange device 10 includes a base 11 fixed with a stepper motor 12, wherein the stepper motor 12 is connected to a first linear slide 13 located above, with the first linear slide 13 being set up in a standing position relative to the base 11. The stepper motor 12 is adapted to drive the first linear slide 13 to rotate along its axis. The medium exchange device 10 further includes a first slider 14 and a second slider 15 slidably mounted on the first linear slide 13. The first slider 14 is equipped with a lower clamping arm 16, and the second slider 15 is equipped with an upper clamping arm 17. The first slider 14 and the second slider 15 can be controlled electronically to move up and down along the same straight line. Additionally, the lower clamping arm 16 and the upper clamping arm 17 perform opening and closing gripping actions under electronic control. A medium exchange container 20 includes a medium exchange cylinder 21, a push rod 22, and an extension tube 23. A communicating tube 211 is formed at an end of the medium exchange cylinder 21, with the extension tube 23 fitting around the communicating tube 211. A wide opening 212 is formed at another end of the medium exchange cylinder 21, allowing the push rod 22 to pass through the wide opening 212 into the medium exchange cylinder 21. The medium exchange cylinder 21 has a capacity of 200 cc to 400 cc for filling culture medium. The lower clamping arm 16 is used to grip the medium exchange cylinder 21, while the upper clamping arm 17 is used to grip the push rod 22. The lower clamping arm 16 sliding along the first linear slide 13 enables positional changes of the medium exchange container 20, while the upper clamping arm 17 sliding along the first linear slide 13 enables suction and discharge of culture medium from the medium exchange container 20. In actual use, when both the lower clamping arm 16 and the upper clamping arm 17 move towards a cell culture bottle 50, the extension tube 23 would enter the cell culture bottle 50 for medium exchange. The used medium exchange container 20 can be replaced through the medium exchange device 10. A medium providing bottle 30 has an opening 31 through which the extension tube 23 can be inserted into the medium providing bottle 30. The upper clamping arm 17 can independently move upward to drive the push rod 22 and the medium exchange cylinder 21 to extract new culture medium from the medium providing bottle 30, filling it into the medium exchange cylinder 21. A recycling bottle 40 has a bottle opening 41 through which the extension tube 23 can be inserted into the recycling bottle 40. The upper clamping arm 17 can independently move downward to drive the push rod 22 and the medium exchange cylinder 21 to infuse old culture medium into the recycling bottle 40. The medium providing bottle 30 and the recycling bottle 40 both have functions for heating and weighing culture medium. They can form seals and have inward suction capabilities at the opening 31 and the bottle opening 41 to prevent culture medium leakage onto the medium exchange device 10.

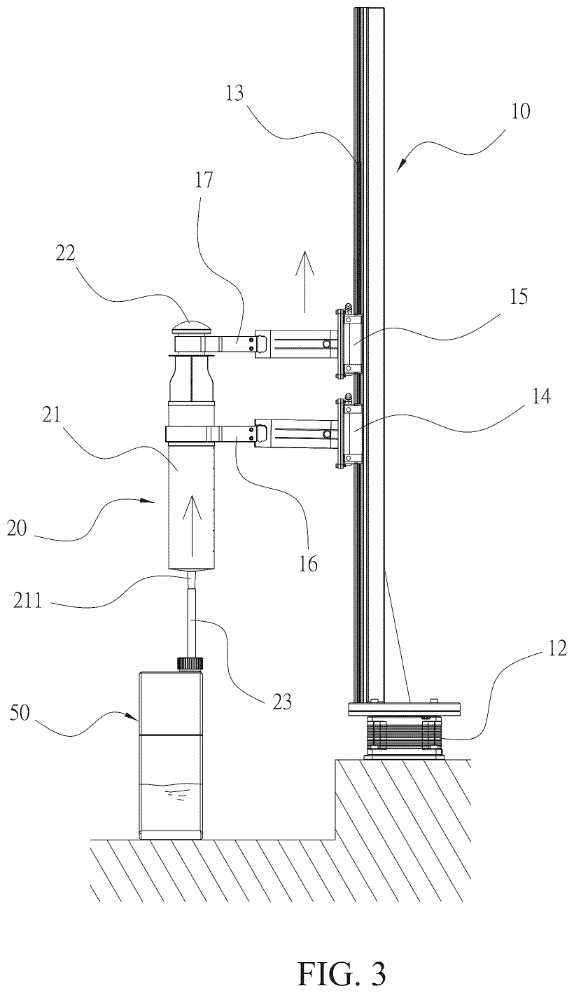

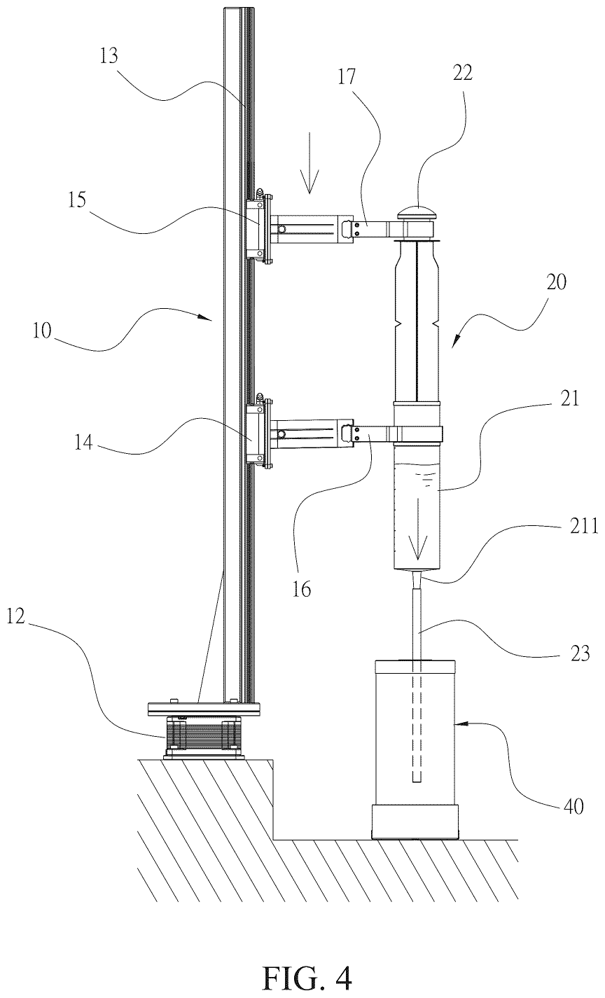

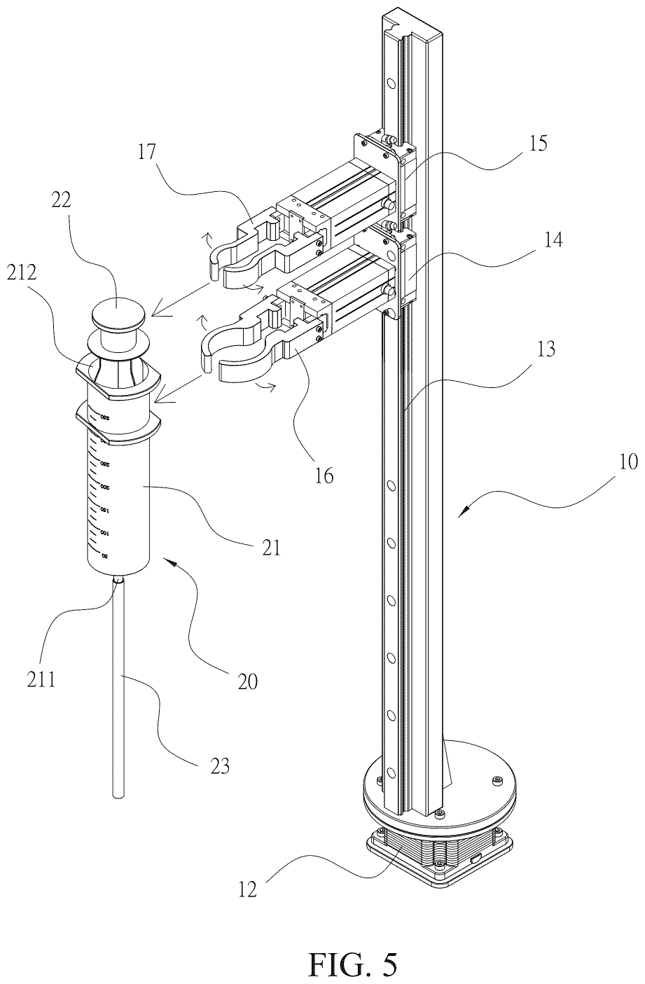

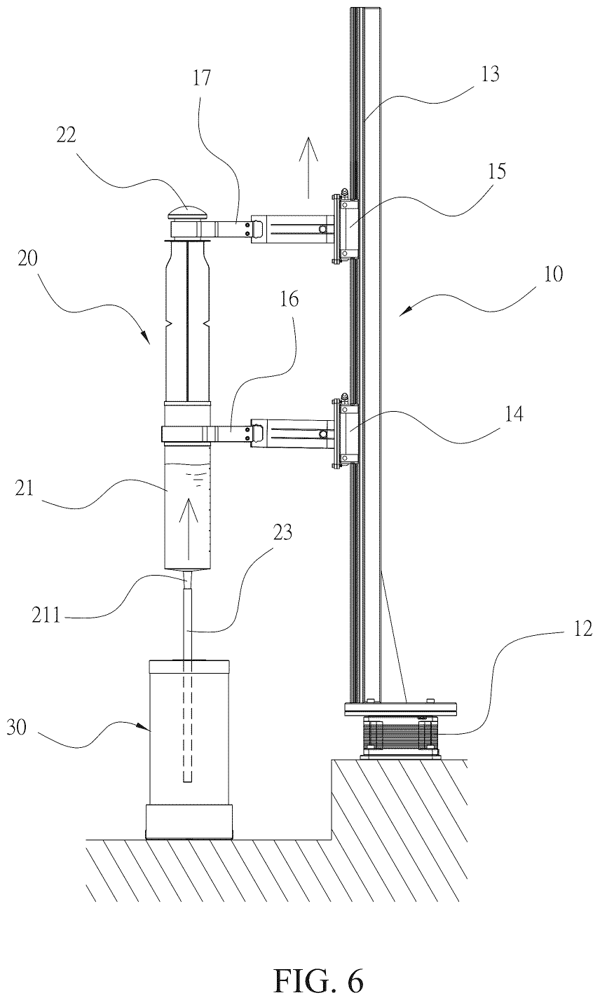

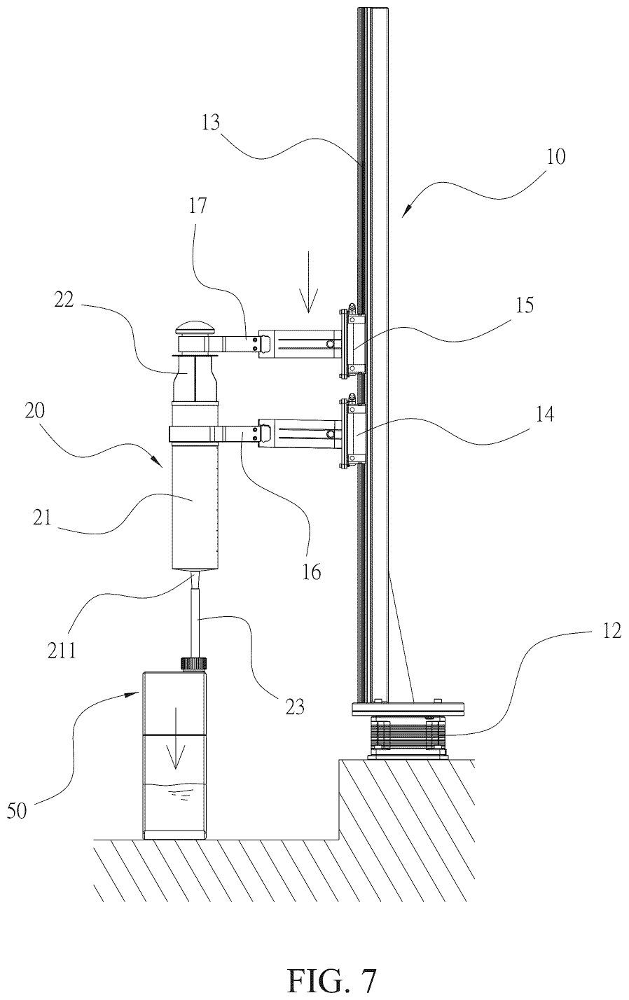

For further explanation, please refer to FIG. 1 through FIG. 7. When culturing cells in the incubator using the cell culture bottle 50, it is necessary to periodically retrieve it to check the cell status and quantity. Before reaching the harvest criteria, the cell culture bottle 50 needs to undergo medium exchange. Firstly, the cell culture bottle 50 is transported to the medium exchange device 10. The medium exchange device 10, driven by the stepper motor 12, rotates the first linear slide 13 to the location where the medium exchange container 20 is stored. Then, the lower clamping arm 16 and the upper clamping arm 17 grip the new medium exchange container 20 (as shown in FIG. 5). The lower clamping arm 16 holds the medium exchange cylinder 21, and the upper clamping arm 17 holds the push rod 22. The first slider 14 and the second slider 15 are then driven downward synchronously along the first linear slide 13, with the first slider 14 controlling the lower clamping arm 16 and the second slider 15 controlling the upper clamping arm 17. This allows the extension tube 23 of the medium exchange container 20 to enter the cell culture bottle 50. Then, the second slider 15 and the upper clamping arm 17 move upward to retract the push rod 22 away from the medium exchange cylinder 21, creating a vacuum suction inside the medium exchange cylinder 21 to extract the old culture medium from the cell culture bottle 50. Once extraction is completed, the first linear slide 13 rotates to position the medium exchange container 20 above the recycling bottle 40. The medium exchange container 20 is lowered to allow the extension tube 23 to enter the recycling bottle 40 through the bottle opening 41, and then the push rod 22 is pressed down to inject the old culture medium from the medium exchange cylinder 21 into the recycling bottle 40. The used medium exchange container 20 is then directly discarded, completing the step of old culture medium recovery. Subsequently, the stepper motor 12 rotates the lower clamping arm 16 and the upper clamping arm 17 to grip a new medium exchange container 20 at a designated position. The next step involves rotating to the medium providing bottle 30, allowing the extension tube 23 to enter the medium providing bottle 30 through the opening 31. The upper clamping arm 17 moves upward to extract a specified amount of culture medium. Finally, rotating back to the cell culture bottle 50 and inserting the extension tube 23, the push rod 22 is pressed into the medium exchange cylinder 21 to inject the new culture medium into the cell culture bottle 50. The used medium exchange container 20 is then directly discarded, completing the step of filling with new culture medium.

In a brief summary, the stepper motor 12 rotates to drive the first linear slide 13 in circular motion, enabling the lower clamping arm 16 and the upper clamping arm 17 to rotate to specified positions for gripping the new and discarding the old medium exchange containers 20. Additionally, they rotate to predetermined positions for extracting old culture medium for recycling and extracting new culture medium for filling. Moreover, the first slider 14 is connected to the lower clamping arm 16, and the second slider 15 is connected to the upper clamping arm 17. The lower clamping arm 16 grips the medium exchange cylinder 21 of the medium exchange container 20, while the upper clamping arm 17 grips the push rod 22. When both the lower clamping arm 16 and the upper clamping arm 17 move up and down simultaneously, they synchronize the vertical movement of the medium exchange container 20. Furthermore, when only the upper clamping arm 17 moves, the push rod 22 is actuated to extract or discharge culture medium, thereby achieving the purpose of replacing culture medium. The used medium exchange container 20 can be directly discarded and replaced with a new one, further preventing mutual contamination between the medium providing bottle 30, the recycling bottle 40, and the cell culture bottle 50, thus combining automatic medium exchange with the practical effect of eliminating contamination risks.

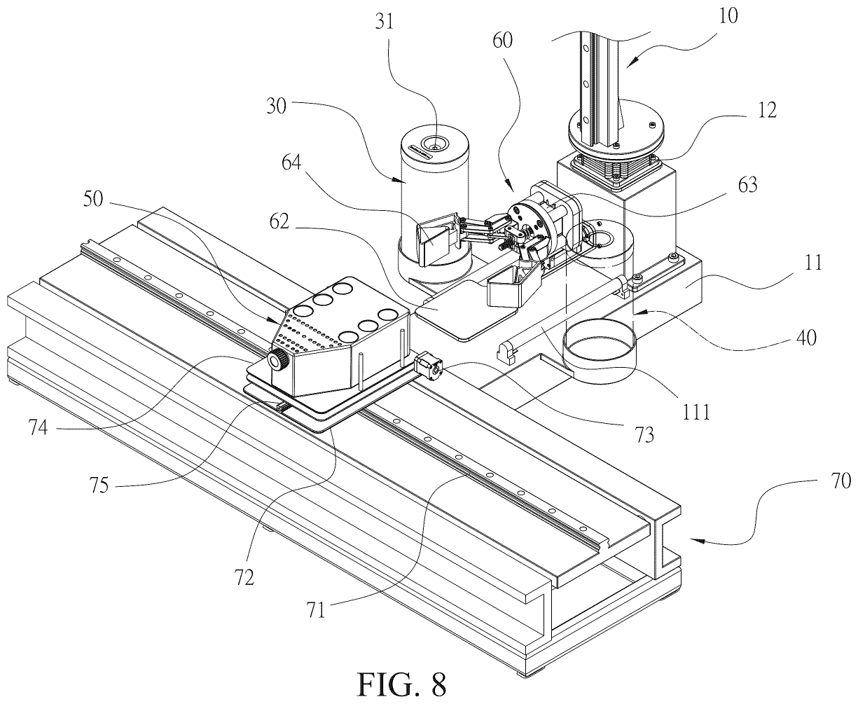

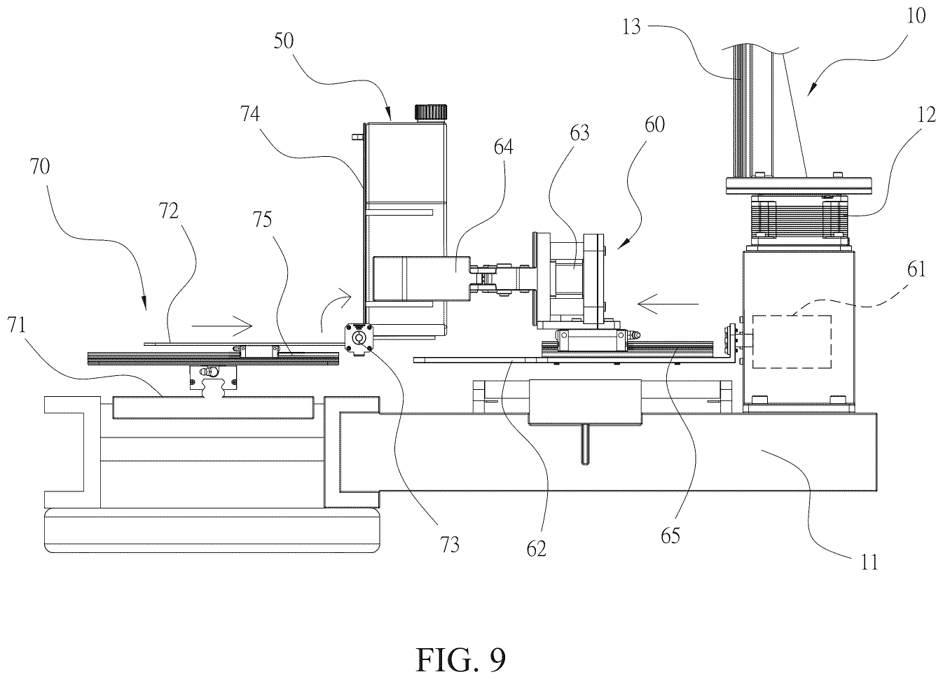

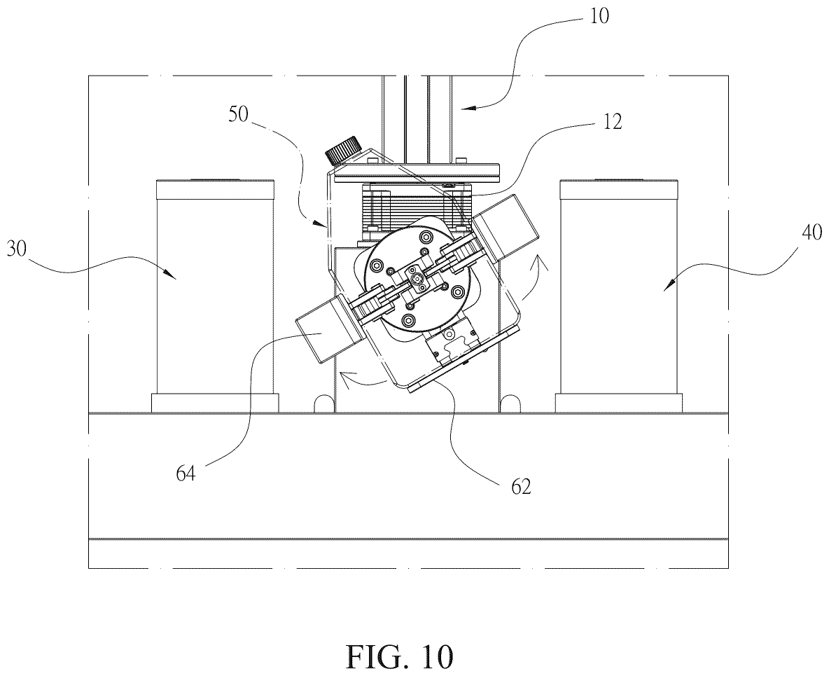

Further, please refer to FIGS. 8, 9, and 10. The present invention additionally includes a gripping mechanism 60, which includes a swing-functioning motor 61, a swing base 62, an extend-functioning motor 63, and a gripping arm 64. The swing-functioning motor 61 is fixed to the base 11 of the medium exchange device 10, and it drives the swing base 62 to enable lateral swinging motion. Additionally, the swing base 62 is equipped with the extend-functioning motor 63, which drives the gripping arm 64 to perform opening and closing gripping actions. The gripping arm 64 is used to hold the cell culture bottle 50 in a specified position, allowing the medium exchange container 20 to exchange medium with the cell culture bottle 50. After medium exchange, the swing-functioning motor 61 drives the cell culture bottle 50 to swing for mixing the culture medium. Moreover, the present invention further includes a transporting mechanism 70, which includes a second linear slide 71. A third linear slide 65 is provided between the swing base 62 and the extend-functioning motor 63. The second linear slide 71 and the third linear slide 65 are arranged perpendicular to each other, with the third linear slide 65 moving the gripping arm 64 to the second linear slide 71 to grip and place the cell culture bottle 50. Furthermore, the second linear slide 71 is equipped with a transporting platform 72, which is provided with a miniature motor 73. The miniature motor 73 drives a flipping board 74, where the cell culture bottle 50 lies horizontally for transportation. When approaching the gripping arm 64, the miniature motor 73 tilts the flipping board 74 to make the cell culture bottle 50 stand upright, enabling the gripping arm 64 to directly grip the standing cell culture bottle 50. Additionally, a fourth linear slide 75 is installed between the transporting platform 72 and the miniature motor 73, perpendicular to the second linear slide 71, allowing the flipping board 74 and the cell culture bottle 50 to move towards the gripping arm 64.

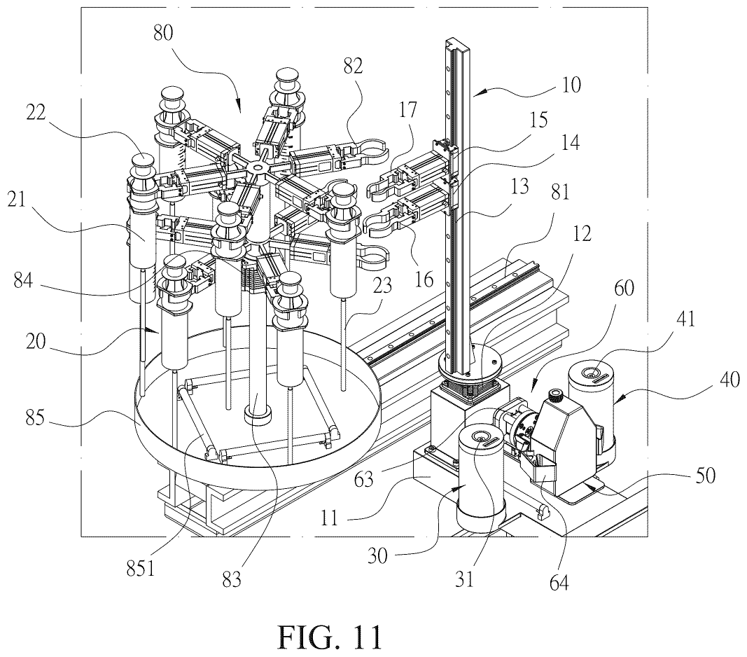

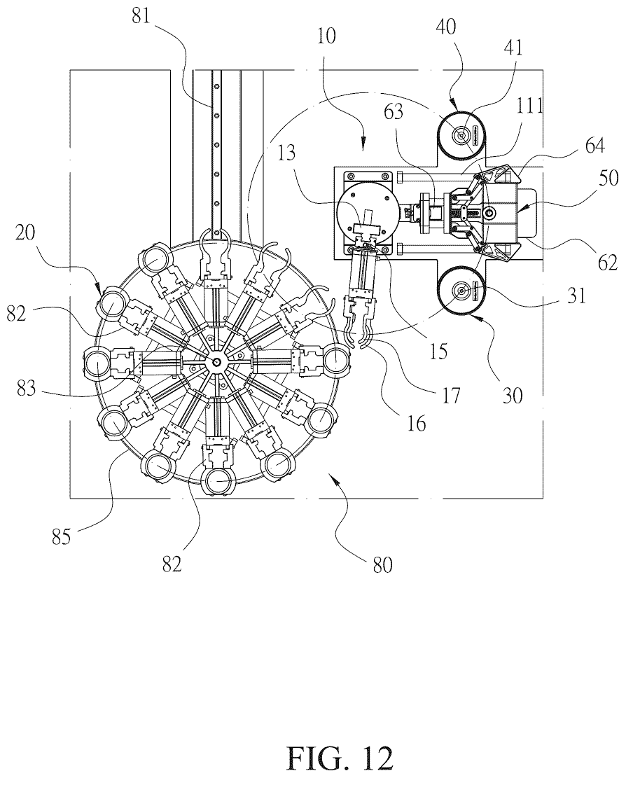

Furthermore, please refer to FIGS. 11 and 12. The present invention additionally includes a container exchange mechanism 80, which includes multiple clamps 82 used for gripping the medium exchange cylinder 21 of the medium exchange container 20. The container exchange mechanism 80 is mounted on a fifth linear slide 81, allowing it to simultaneously meet the demand for replacing the medium exchange container 20 of multiple sets of the medium exchange device 10. After the clamp 82 grips the medium exchange container 20 and rotates it to the designated position, it provides the lower clamping arm 16 and the upper clamping arm 17 of the medium exchange device 10 to grip the medium exchange container 20. Alternatively, the unclamped clamps 82 can also provide storage for the used medium exchange container 20, continuously replenishing and recycling the medium exchange container 20. This ensures that the medium exchange device 10 replaces the medium exchange container 20 with a new one after each extraction and infusion, effectively preventing mutual contamination between multiple cell culture bottles 50. To elaborate further, the multiple clamps 82 are radially arranged around a center post 83, allowing all the medium exchange containers 20 to surround the center post 83 in a circular manner. The clamps 82 can form multiple layers according to the quantity requirements. Additionally, a driving motor 84 is installed at the center post 83 to rotate it, facilitating the transfer of the medium exchange containers 20 between the clamps 82 and the lower clamping arm 16. By the fifth linear slide 81, the container exchange mechanism 80 is equipped with a drip receiving disk 85, and at least one UV lamp 851 (111) is installed on both the drip receiving disk 85 and the base 11 of the medium exchange device 10. The drip receiving disk 85 collects dripping culture medium and the UV lamp 851 (111) simultaneously sterilizes both the container exchange mechanism 80 and the medium exchange device 10.

It should be realized that the above description is only some preferred embodiments of the present invention and should not be deemed as limitations of implementing the present invention. All substantially equivalent variations and modifications which employ the concepts disclosed in this specification and the appended claims should fall within the scope of the present invention.

What is claimed is:

1. A medium exchange system for cell culture bottles that prevents mutual contamination, comprising:
a medium exchange device, which has a stepper motor fixed to a base thereof, wherein the stepper motor is connected to a first linear slide located above, and is adapted to drive the first linear slide to rotate; the medium exchange device is provided with a first slider and a second slider in a slidable manner at the first linear slide; the first slider is provided with a lower clamping arm, and the second slider is provided with a upper clamping arm; and
a medium exchange container, which comprises a medium exchange cylinder, a push rod, and an extension tube, wherein the medium exchange cylinder has a communicating tube formed at an end thereof; the extension tube fits around the communicating tube; a wide opening is formed at another end of the medium exchange cylinder, whereby the push rod enters the medium exchange cylinder through the wide opening; the lower clamping arm is adapted to clamp the medium exchange cylinder, and the upper clamping arm is adapted to clamp the push rod; the lower clamping arm and the upper clamping arm are both adapted to move toward a cell culture bottle, so that the extension tube is adapted to enter the cell culture bottle for medium exchange, and the medium exchange device is adapted to replace the used medium exchange container.

2. The medium exchange system of claim 1, further comprising a medium providing bottle, wherein the medium providing bottle has an opening provided thereon, and the medium exchange container is adapted to be placed into the medium providing bottle by passing the extension tube through the opening; when only the upper clamping arm moves upward to move the push rod and the medium exchange cylinder to extract new culture medium in the medium providing bottle, the new culture medium is filled into the medium exchange cylinder.

3. The medium exchange system of claim 2, further comprising a recycling bottle, wherein the recycling bottle has a bottle opening provided thereon, and the medium exchange container is adapted to be placed into the recycling bottle by passing the extension tube into the bottle opening; when only the upper clamping arm moves downward to move the push rod and the medium exchange cylinder, the old culture medium is injected into the recycling bottle.

4. The medium exchange system of claim 1, further comprising a gripping mechanism, wherein the gripping mechanism comprises a swing-functioning motor, a swing base, a extend-functioning motor, and a gripping arm, wherein the swing-functioning motor is fixed to the base of the medium exchange device, and the swing-functioning motor is linked to the swing base to drive the swing base; the extend-functioning motor is provided at the swing base, and the extend-functioning motor is linked to the gripping arm to drive the gripping arm, so that the gripping arm is adapted to grip the cell culture bottle in this manner, and the swing-functioning motor is adapted to shake the cell culture bottle for mixing culture medium.

5. The medium exchange system of claim 4, further comprising a transporting mechanism, wherein the transporting mechanism is provided with a second linear slide; a third linear slide is provided between the swing base and the extend-functioning motor; the second linear slide and the third linear slide are perpendicular to each other; the third linear slide drives the gripping arm to move to the second linear slide for gripping and placing the cell culture bottle.

6. The medium exchange system of claim 5, wherein a transporting platform is provided at the second linear slide in a slidable manner; the transporting platform is provided with a miniature motor, and the miniature motor is linked to a flipping board to drive the flipping board; the cell culture bottle is adapted to lie on the flipping board for transportation; when the cell culture bottle is transported to the gripping arm, the miniature motor drives the flipping board to erect the cell culture bottle.

7. The medium exchange system of claim 6, wherein a fourth linear slide is provided between the transporting platform and the miniature motor, and the fourth linear slide and the second linear slide are perpendicular to each other, so that the flipping board and the cell culture bottle are both movable toward the gripping arm.

8. The medium exchange system of claim 1, further comprising a container exchange mechanism, wherein the container exchange mechanism is provided on a fifth linear slide in a slidable manner, and the container exchange mechanism is provided with a plurality of clamps, wherein the clamps are adapted to clamp the medium exchange cylinder of the medium exchange container, and provide the medium exchange container to the lower clamping arm and the upper clamping arm of the medium exchange device.

9. The medium exchange system of claim 8, wherein the clamps are provided at a center post in a radial pattern, so that all of the medium exchange containers surround the center post; a driving motor is provided at the center post, wherein the driving motor is adapted to rotate the center post, whereby the clamps and the lower clamping arm are adapted to deliver the medium exchange container to each other.

10. The medium exchange system of claim 9, wherein the container exchange mechanism has a drip receiving disk provided by the fifth linear slide, and at least an UV lamp is provided both at the drip receiving disk and the base of the medium exchange device.
